(19) United States
(12) Patent Application Publication
(10) Pub. No.: US 2010/0099840 A1
(43) Pub. Date: Apr. 22, 2010

(54) DIOXYPYRROLO-HETEROAROMATIC COMPOUNDS AND ORGANIC ELECTRONIC DEVICES USING THE SAME

(76) Inventors: Jae-Min Moon, Daejeon Metropolitan City (KR); Hyeon Choi, Daejeon Metropolitan City (KR); Jae-Min Lee, Daejeon Metropolitan City (KR)

Correspondence Address: MCKENNA LONG & ALDRIDGE LLP, 1900 K STREET, NW, WASHINGTON, DC, 20006, US

(21) Appl. No.: 12/449,927

(22) PCT Filed: Apr. 11, 2008
(86) PCT No.: PCT/KR2008/002048

§ 371 Date: Sep. 3, 2009

(30) Foreign Application Priority Data
Apr. 13, 2007 (KR) 10-2007-0036542

(51) Int. Cl. C08G 75/00; C07D 495/02

(52) U.S. Cl. 528/321; 548/453

(57) ABSTRACT

The present invention relates to dioxypyrrolo-heterocyclic compounds and an organic electronic device using the same. The compound of the present invention satisfies the requirements for use in an organic electronic device such as an organic light emitting device, an organic thin film transistor, and an organic solar cell, for example, suitable energy levels, and the electrochemical and thermal stability, by introducing various substituents to the core structure, and also have amorphous or crystalline property depending on the Mnd of the substituents, to satisfy the characteristics individually required for each of the devices. Further, an organic semi-conductor of p-type or n-type can be fabricated by introducing various substituents to the core structure having a property of n-type, thereby providing stability for the device.

### TECHNICAL FIELD

The present invention relates to dioxypyrrolo-heterocyclic compounds and an organic electronic device using the same.

This application claims priority benefits from Korean Patent Application No. 10-2007-36542, filed on Apr. 13, 2007, the entire content of which is fully incorporated herein by reference.

### BACKGROUND ART

The current society, also referred to as an information society, has proceeded, accompanied by the discovery of an inorganic semiconductor, which typically includes Si, and by the development of a wide range of the electronic devices using the same. However, the preparation of an electronic device using an inorganic material requires a high temperature or vacuum process, thus needing lots of investments on the equipment for the device. The inorganic material has undesirable physical properties for the flexible displays which are a current focus as a next-generation display.

In order to solve the above-described problems, there are suggested organic semiconductor materials, which have recently attracted a great deal of attentions as a semiconductor material having various physical properties. The organic semiconductor materials can be employed in a variety of the electronic devices, which had previously used inorganic semiconductor materials. Typical examples of the electronic devices using the organic semiconductor materials include an organic light emitting device, an organic thin film transistor, and an organic solar cell.

The organic electronic device such as an organic light emitting device, an organic thin film transistor, and an organic solar cell, is an electronic device employing the semicondutor properties of the organic semiconductor material, and usually comprises at least two electrodes and an organic material layer between the two electrodes. For example, an organic solar cell generates electricity using the electrons and the holes, separated from excitons, which are generated in an organic material layer by means of solar energy. The organic light emitting device introduces the electrons and the holes from two electrodes to an organic material layer, so as to convert the current to visible light. The organic thin film transistor transports the holes or the electrons which are formed on an organic material layer between a source electrode and a drain electrode by means of the voltage applied on a gate. In order to improve the electronic devices, the electronic devices can further comprise an electron/hole injecting layer, an electron/hole extracting layer, or an electron/hole transporting layer.

The organic semiconductor materials that are used for the electronic devices should have good hole or electron mobility. To meet this requirement, most of the organic semiconductor materials have conjugated structures.

Further, the organic semiconductor materials used in each of the electronic devices have various preferable morphologies, depending on the characteristics required from the devices. For example, when a thin film is formed using an organic semiconductor material, the thin film in the organic light emitting device preferably has an amorphous property. That is, if an organic thin film in the organic light emitting device has a crystalline property, light emitting efficiency may be reduced, the quenching sites in the charge transport are increased, or the leakage current is increased, thus leading to deterioration of the device performance.

On the other hand, it is favorable that the organic thin film transistor has larger charge mobility of the organic material layer, and accordingly, it is preferable that packing between the molecules of the organic materials are effected, thereby providing the organic thin film with crystallinity. Most preferably, this crystalline organic film has particularly single crystal, and in the case where this crystalline organic film has a polycrystalline form, preferably, the size of each domain is large, and the domains are well connected with each other.

In order to satisfy these requirements, the substances having non-planar structures such as NPB (4,4′-bis[N-(1-naphthyl)-N-phenylamino]biphenyl), and Alq₃ (aluminum tris(8-hydroxyqinoline)) are usually used so as to form amorphous thin films in the organic light emitting devices; and the substances having rod-like structures such as pentacene, and polythiophene, or the substances having planar structures such as phthalocyanine derivative are usually used so as to easily effect packing between the molecules in the organic thin film transistors.

On the other hand, the organic electronic device can be prepared, which comprises at least two organic material layers, in which at least two kinds of the organic semiconductor material having different applications are laminated, in order to improve the device performance.

For example, the organic light emitting device can further comprise a hole injecting layer, a hole transporting layer, an electron transporting layer, and an electron injecting layer to facilitate injection and transportation of the holes or electrons from the anode or the cathode, thus enhancing the device performance.

In the case of the organic thin film transistor, there is introduced a method, which involves introducing an auxiliary electrode comprising an organic semicondutor, or subjecting an SAM(Self Assembled Monolayer) treatment to the electrode with an organic material to reduce the contact resistance between the semiconductor layer and the electrode. Further, there is used a method, which involves treating the surface of the insulating layer with an organic material or using an organic insulating film to improve the characteristics concerning the contact with a semiconductor consisting of organic material.

In addition, the organic semiconductor material used in the organic electronic device preferably has thermal stability against Joule heat which is generated daring the transfer of the charges in the device, and also preferably has a suitable band gap, and an HOMO (Highest Occupied Molecular Orbital) or LUMO (Lowest Unoccupied Molecular Orbital) energy level for easy injection or transport of the charges. Further, the organic semiconductor material should be excellent in chemical stability, and the electrode should be excellent in the interface characteristics with an adjacent layer, as well as in stability against moisture or oxygen.

There is a need of an organic material, which satisfies the characteristics commonly required. in the organic electronic device as described above, or the characteristics individually required according to the kind of the electronic device, and if desired, which is more suitable for specific applications in the relevant field

### DISCLOSURE OF INVENTION

### Technical Problem

It is an object of the present invention to provide a novel heterocyclic compound comprising a dioxypyrrolo group, which has electrochemical stability and easily functions as an organic semiconductor of p-type or n-type depending on the kind of substituents, and a stable organic electronic device using the same.

### Technical Solution

The present invention provides a compound represented by the following Formula 1, and an organic electronic device using the same.

Hereinafter, the present invention will be described in detail.

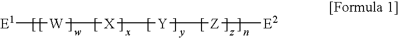

wherein W and Y are the same as or different from each other, and are each independently represented by the following Formula 2,

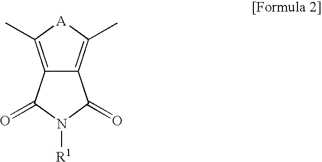

wherein R¹ is the same as or different from each other, and are each independently a hydrogen atom; a halogen atom; an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, R′ and R″ are the same as or different from each other, and are each independently H, F, Cl, or CN, A¹ and A² are the same as or different from each other, and are each independently an alkyl group having 1 to 20 carbon atoms or aryl group,

A is O, S, Se, NR³, SiR³R⁴,or CR³R⁴, wherein R³ and R⁴ are the same as or different from each other, and are each independently a hydrogen atom; an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, and R³ and R⁴ may be bonded to each other to form a ring, R′ and R″ are the same as or different from each other, and are each independently H, F, Cl, or CN, A¹ and A² are the same as or different from each other, and each independently an alkyl group or aryl group having 1 to 12 carbon atoms,

In the Formula 1,

X and Z are the same as or different from each other, and are each independently —CA¹═CA²—; —C≡C—; an arylene group substituted with at least one R²; or a heteroarylene group substituted with at least one R², R² is the same as or different from each other, and is each independently a hydrogen atom; a halogen atom; an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, R′ and R″ are the same as or different from each other, and are each independently H, F, Cl, or CN, A¹ and A² are the same as or different from each other, and are each independently an alkyl group having 1 to 12 carbon atoms or aryl group,

E¹ and E² are the same as or different from each other, and are each independently, a hydrogen atom; a halogen atom; an aryl group; a heteroaryl group; —Sn(R′R″R′″)₃; —B(OR′)(OR″); —CH₂Cl; —CHO; —CH═CH; —SiR′R″R′″,

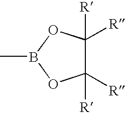

or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, R′, R″ and R′″ are the same as or different from each other, and each independently H, F, Cl, or CN, A¹ and A² are the same as or different from each other, and each independently an alkyl group having 1 to 12 carbon atoms or aryl group,

w, x, y, and z are mole fractions of W, X, Y, and Z, respectively,

w is a real number satisfying 0<w≦1

x is a real number satisfying 0≦x<1,

y is a real number satisfying 0≦y<1,

z is a real number satisfying 0≦z<1, w+x+y+z=1, and

n is an integer of 1 to 10,000,

with the proviso that w=1, x=y=z=0, and when R¹ is an alkyl group, E¹ and E² are not hydrogen atom or halogen atom.

### ADVANTAGEOUS EFFECTS

The compound of the present invention is a novel compound, which satisfies the requirements for being used in an organic electronic device such as an organic light emitting device, an organic thin film transistor, and an organic solar cell, for example, suitable energy levels, and the electrochemical and thermal stability, by introducing various substituents to the core structure, and also have amorphous or crystalline property depending on the kind of the substituents, to satisfy the characteristics individually required for each of the devices. Further, an organic semiconductor of p-type or n-type can be fabricated by introducing various substituents to the core structure having a property of n-type, thereby providing stability for the device. Therefore, the compound of the present invention can play various roles in the organic electronic device, and provide a device having higher charge mobility and stability when it is employed in the organic electronic device.

### BRIEF DESCRIPTION OF THE DRAWINGS

FIG. 1 illustrates an example of the organic light emitting device comprising a substrate 1, anode 2, a hole injecting layer 3, a hole transporting layer 4, a light emitting layer 5, an electron transporting layer 6, and a cathode 7;

FIG. 2 illustrates an example of the organic thin film transistor device of a bottom contact type, comprising a substrate 8, an insulating layer 9, a gate electrode 10, a source electrode 11, a drain electrode 12, and an organic material layer 13;

FIG. 3 illustrates an example of the organic thin film transistor device of a top contact type, comprising a substrate 8, an insulating layer 9, a gate electrode 10 a source electrode 11, a drain electrode 12, and an organic material layer 13;

FIG. 4 illustrates an example of the organic solar cell comprising a substrate 14, an anode 15, an electron donor layer 16, an electron acceptor layer 17, and a cathode 18; and

FIGS. 5 and 6 are each a graph showing properties of the transistor prepared in Example 2.

### BEST MODE FOR CARRYING OUT THE INVENTION

In Formula 1, A is preferably S.

The compound represented by Formula 1 preferably has E¹ and E² of same functional group.

In the compound represented by Formula 1, n is preferably 2 to 5,000, more preferably 10 to 5,000, and most preferably 20 to 1,000.

The compound represented by Formula 1 preferably has a molecular weight of 1,000 to 500,000, and more preferably has a molecular weight of 5,000 to 300,000. In the case where the molecular weight is less than 1,000, it is difficult to apply the above material on the surface during solution process. In the case where the molecular weight is more than 500,000, the solubility in a solvent is reduced, and a thin film is difficult to be formed

In the repeating unit of

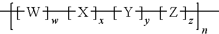

which is present in the compound represented by Formula 1, the kind and repeating number of each monomer W, X, Y, and Z may be the same or different from each other. Therefore, each monomer may be a homopolymer or copolymer, and examples thereof are as follows:

Random copolymer: A polymer in which the molecules of each monomer are randomly arranged in the polymer backbone, such as -W-X-Y-Y-Z-W- or -W-X-W-X-X-.

Alternating copolymer: A polymer formed of two different monomer molecules that alternate in sequence in the polymer chain, such as -W-X-W-X-W-X-, -W-X-Y-W-X-Y-, or -W-X-Y-Z-W-X-Y-Z-.

Block copolymer: A polymer in which the like monomer units occur in relatively long alternate sequences on a chain, such as -W-W-W-X-X-Y-Y-Y-Z-Z-Z-.

The compound represented by Formula 1 according to the present invention is preferably an alternating copolymer consisting of same repeating units in the form of [W-X]ₙ, [W-X-Y]ₙ, [W-X-Z]ₙ, [W-Y-Z]ₙ, or [W-X-Y-Z]ₙ.

In Formula 1, in the case where X and Z are arylene groups or heteroarylene groups, the arylene group or heteroarylene group has preferably one to three rings of 25 or less carbon atoms, in which the rings may be fused. The heteroarylene group contains at least one heteroatom, preferably a nitrogen atom, oxygen atom, sulfur atom or selenium atom. The arylene group or heteroarylene group may be substituted with at least one of F, Cl, Br, I, or CN, and may be substituted with a straight, branched, or cyclic alkyl group having 1 to 20 carbon atoms which is unsubstituted or substituted with F, Cl, Br, I, or CN, and CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, in which R′ and R″ are the same as or different from each other and are each independently H, F, Cl, or CN, and A¹ and A² are the same as or different from each other and each independently an alkyl group or aryl group having 1 to 12 carbon atoms.

Preferred examples of the arylene group or heteroarylene group include a phenylene group, a phenylene group substituted with at least one nitrogen atom, a naphthalene group, an alkyl fluorine group, an oxazole group, a thiophene group, a selenophene group, or a dithienothiophene group, and may be substituted with at least one hydrogen atom; aryl group; heteroaryl group; a straight, branched, or cyclic alkyl group having 1 to 20 carbon atoms which is unsubstituted or substituted with F, Cl, Br, I, or CN, and CH groups in the alkyl group which are not adjacent to each other may be each independently unsubstituted or substituted with —O—, —S—, —NH—, —NR′—, —SiR′R″—, —CO—, —COO—, —OCO—, —O—CO—O—, —S—CO—, —CO—S—, —CA¹═CA²—, or —C≡C—, in which R′ and R″ are the same as or different from each other and are each independently H, F, Cl, or CN, and A¹ and A² are the same as or different from each other, and are each independently an alkyl group having 1 to 12 carbon atoms or aryl group.

Examples of X and Z is described as follows. However, the following Examples are presented for the purpose of illustrating the present invention, and are not limited thereto.

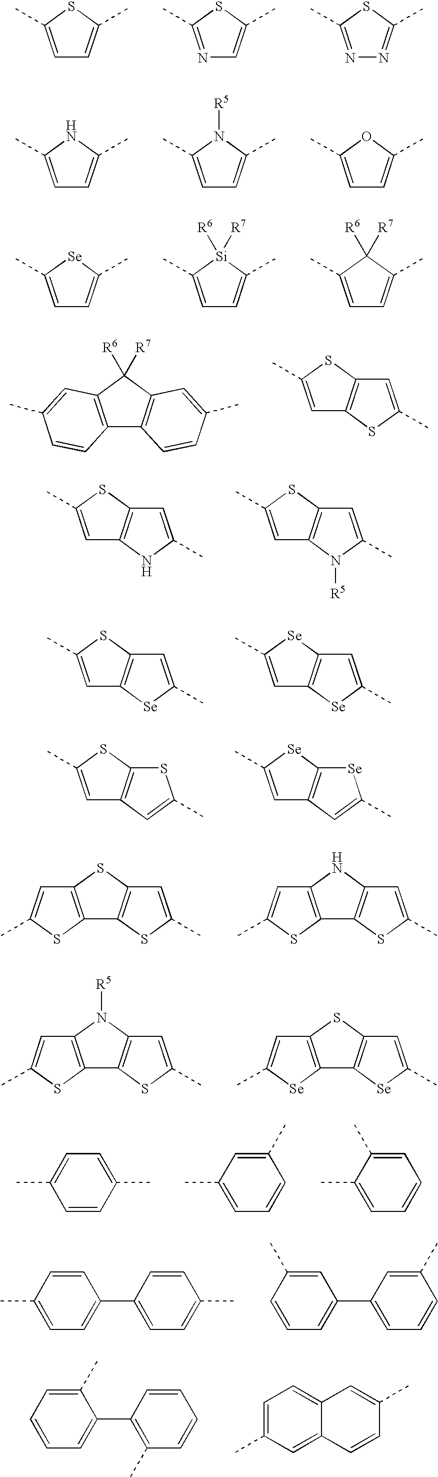

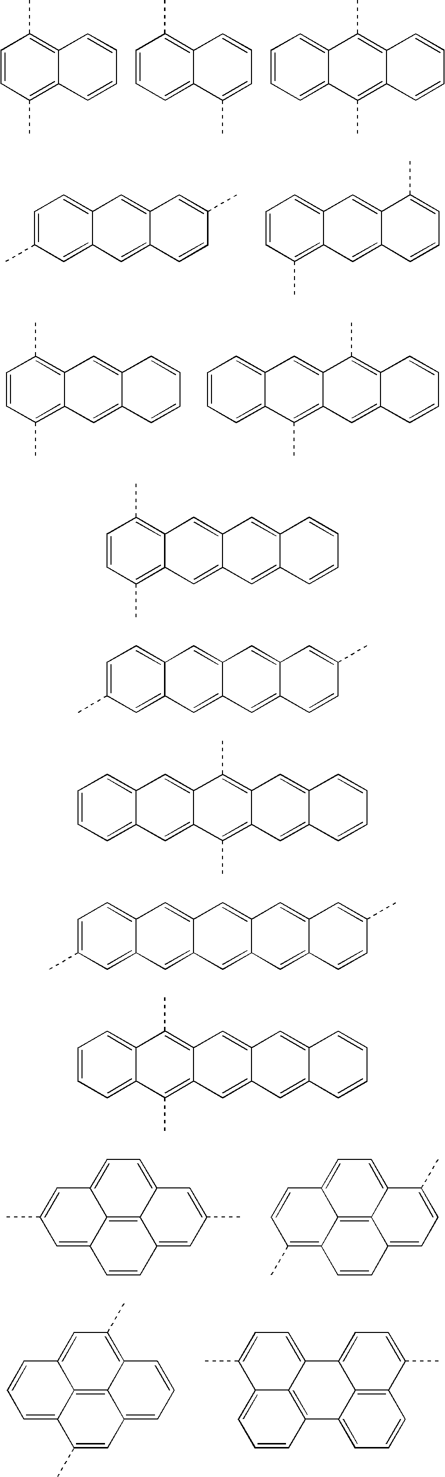

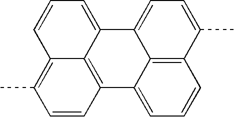

In the structural formulae, the arylene group or heteroarylene group may substituted with at least one selected from the group consisting of a halogen group, an alkyl group, an alkoxy group, a thioalloxy group, an aryl group, an amino group, a nitrile group, a nitro group, an ester group, an ether group, an amide group, an imide group, a hetero group, a vinyl group, an acetylene group, and a silane group, and R⁵, R⁶, and R⁷ are the same as or different from each other and are each independently a hydrogen atom, alkyl group, or aryl group.

In Formula 1, in the case where R¹ and R² are aryl group or heteroaryl group, the arylene group or heteroarylene group has preferably one to three rings of 25 or less carbon atoms, in which the rings may be fused The heteroarylene group contains at least one heteroatom, preferably a nitrogen atom, oxygen atom, sulfur atom or selenium atom. R¹ and R² may be substituted with at least one of F, Cl, Br, I, or CN, and may be substituted with a straight, branched, or cyclic alkyl group having 1 to 20 carbon atoms which is unsubstituted or substituted with F, Cl, Br, I, or CN, and CH₂ groups in the alkyl group which are not adjacent to each other may be each independently substituted with —O—, —S—, —NH—, —NR′—, —SiR′R″—, —CO—, —COO—, —OCO—, —O—CO—O—, —S—Co—, —Co—S—, —CA′═CA²—, or —C≡C—, in which R′ and R″ are the same as or (Afferent from each other and are each independently H, F, Cl, or CN, and A¹ and A² are the same as or different from each other, and are each independently an alkyl group having 1 to 12 carbon atoms or aryl group.

In Formula 1, examples of the aryl group of R¹, R², R³, R⁴, E¹, and E² include a phenyl group, a naphthyl group, an anthracenyl group, a pyrenyl group, a pherylenyl group, a pyridyl group, a bipyridyl group, a carbazle group, a thiophenyl group, a quinolinyl group, and an isoqtinolinyl group, but are not limited thereto.

In Formula 1, the heteroaryl group of R¹, R², R³, R⁴, E¹, and E² is an aryl group having a ring substituted with at least one heteroatom, and examples thereof include a furyl group, a pyridyl group, a pyrrolyl group, and a phenanthryl group, but are not limited thereto.

In one preferred embodiment of the present invention, specific example of Formula 1 is represented by the following Formulae. However, the following Examples are presented for the purpose of illustrating the present invention, and are not limited thereto.

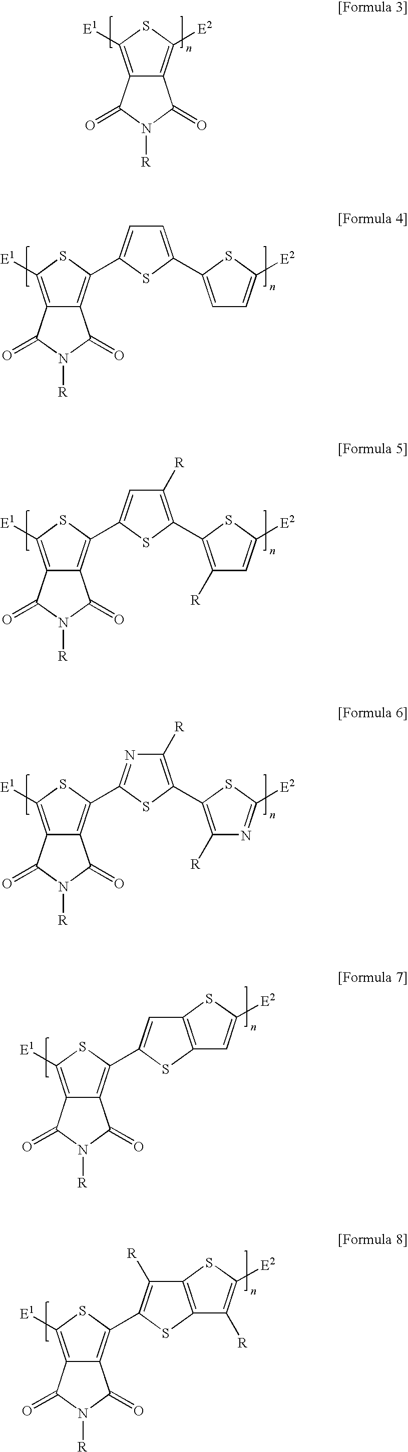

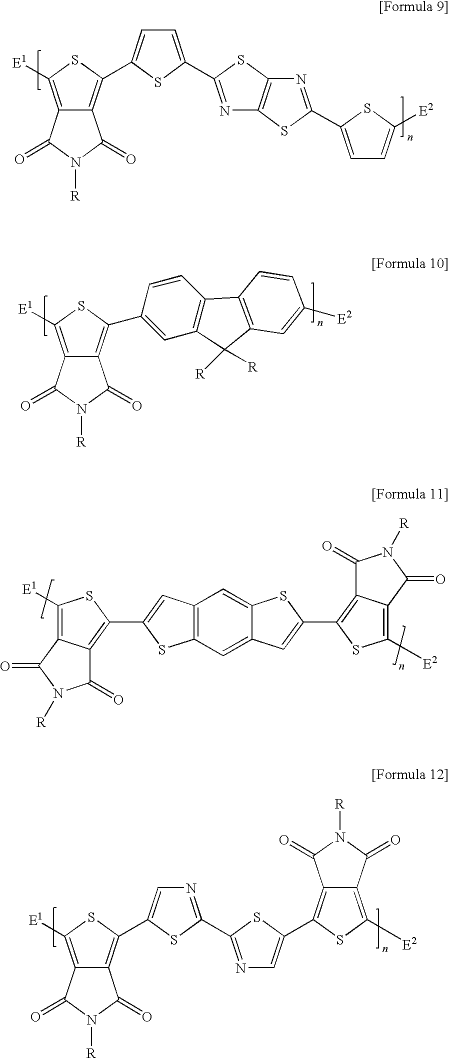

In Formulae 3 to 12,

Rs are the same as defined in R¹ of Formula 1, and may be the same as or different from each other in the same molecule,

n, E¹, and E² are the same as defined in Formula 1.

The halogen substituents of W and Y for the preparation of the compound of Formula 1 may be generally prepared by the following Reaction Scheme 1.

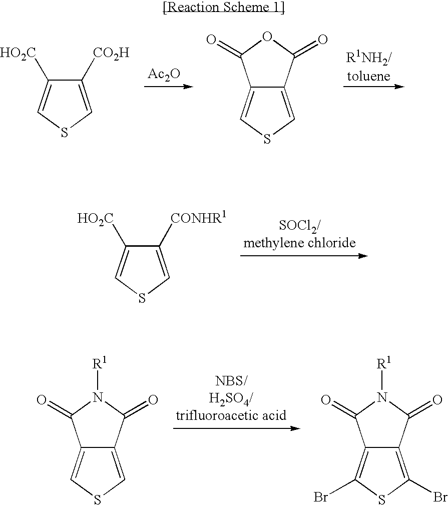

Wherein R¹ is the same as defined in Formula 1, bromine is an example of halogen atom, and fluoride, chloride, iodine or the like may be used

Subsequently, the prepared halogen substituent of W or Y in Formula 1 may react with a substance having a structure represented by X or Z by the method such as Stille coupling, Kumala coupling, and Suzuki coupling, to prepare a polymer material.

Typical examples of the organic electronic devices using the organic semiconductor which is prepared by using the compound represented by Formula 1 according to the present invention include an organic light emitting device, an organic thin film transistor, and an organic solar cell. The organic semiconductor is used in the organic electronic device, and includes n-type and p-type semiconductors like inorganic semi conductor.

For example, in the organic light emitting device, the p-type semiconductor is used as a hole injecting layer or hole transporting layer, and the n-type semiconductor is used as an electron transporting layer or electron injecting layer. Further, the semiconductor used as a light emitting layer should be stable for electrons and holes, and may include all structure showing n and p type properties. Further, it is preferable that the organic light emitting device has a structure minimizing the packing between the molecules, as described above.

On the other hand, in the organic thin film transistor, a p-type semiconductor, in which a charge induced by a gate voltage is a hole, and an n-type semiconductor, in which a charge induced by a gate voltage is an electron, are used, and both of them are used in one device to fabricate a transistor having ambipolarity, thus reducing current consumption. However, among the organic semiconductors for the transistor that have been known up to now, the p-type semiconductor has been known to have better properties and higher stability. In order to solve the problems, a material having the properties of n-type and p-type is introduced to a molecule to increase the stability or may be used as the ambipolar material. Further, the organic thin film transistor preferably has a structure having the good packing between molecules in order to increase charge mobility.

In the compound represented by Formula 1, W and Y containing the dioxypyrrolo group is a structure that functions to withdraw electrons in hetero ring and has the property of n-type, and the derivatives, in which suitable substituents are introduced to the group, can be employed in an electron injecting layer or an electron transporting layer of the organic light emitting device. Since the structure is capable of forming hydrogen bond, the substituents inducing the good pacing are intruded to X or Z of Formula 1 to be used as a semiconductor layer of the organic thin film transistor. The structure functions as an n-type semiconductor. In the case where as a substituent, the p-type substituent is introduced to induce the ambipolar semiconductor or in the case where the stronger p-type substituent is introduced, the organic semiconductor material, which maintains the property of p-type semiconductor and has the improved stability, can be obtained

Accordingly, the compound represented by Formula 1 of the present invention have suitable properties as an organic semiconductor material used for the organic electronic device such as an organic light emitting device, an organic thin film transistor, and an organic solar cell.

Meanwhile, in the present invention, the organic electronic device contains two or more electrodes and one or more organic material layers disposed between two electrodes, in which one or more of the organic material layers comprise the compound of Formula 1. The organic electronic device may be an organic light emitting device, an organic thin film transistor, or an organic solar cell.

The compound represented by Formula 1 can be applied to the organic electronic device by either vacuum deposition or solution coating. In particular, in the case of the derivative having high molecular weight, a thin film having excellent film quality can be obtained by the method of solution coating.

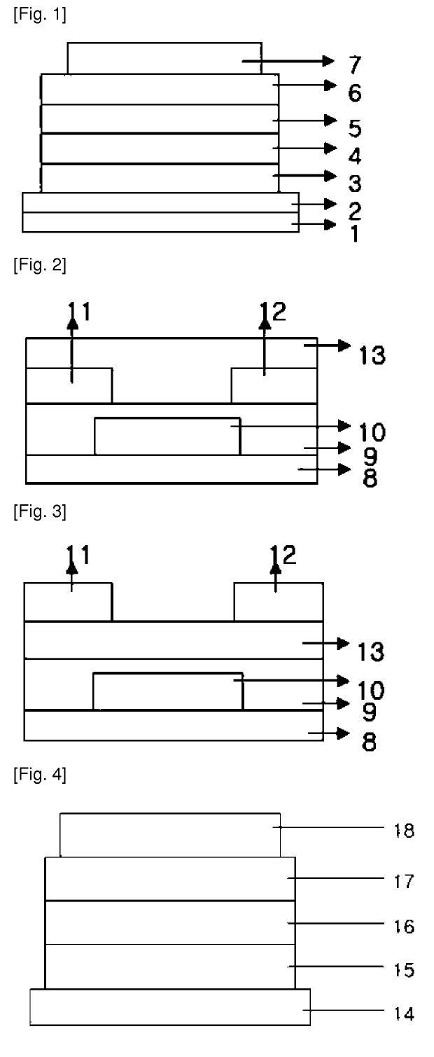

In the case where the organic electronic device according to the present invention is the organic light emitting device, the organic electronic device may have a structure in which a first electrode, one or more organic material layers, and a second electrode are sequentially stacked The organic material layer may be a multilayered structure comprising at least two layers selected from hole injecting layer, hole transporting layer, light emitting layer, electron transporting layer or the like, but are not limited thereto, may be a monolayered structure. An example of the organic light emitting device according to the present invention is illustrated in FIG. 1. For example, the organic light emitting device according to the present invention can be prepared by using a PVD (physical vapor deposition) process such as sputtering and e-beam evaporation or solution coating. The organic light emitting device can be prepared by depositing a metal, a metal oxide having conductivity or an alloy thereof on a substrate 1 to form an anode 2; forming an organic material layer comprising a hole injecting layer 3, a hole transporting layer 4, a light emitting layer 5, and an electron transporting layer 6 on the anode; and depositing a cathode 7 thereon. Alternatively, the organic light emitting device can be prepared by sequentially depositing a cathode material, an organic material layer, and an anode material on a substrate.

If the organic electronic device according to the present invention is an organic thin film transistor, it may have the structure as shown in FIG. 2 or 3. That is, the organic thin film transistor according to the present invention may have the structure comprising a substrate 8, an insulating layer 9, a gate electrode 10, a source electrode 11, a drain electrode 12, and an organic material layer 13. The organic material layer in the organic thin film transistor of the present invention can be formed in the structure of a monolayer or a multilayer.

If the organic electronic device according to the present invention is an organic solar cell, its structure may be a structure as shown in FIG. 4. That is, the organic solar cell according to the invention may have a structure in which a substrate 14, an anode 15, an electron donor layer 16, an electron acceptor layer 17, and a cathode 18 are sequentially stacked

### MODE FOR THE INVENTION

Hereinafter, the present invention will be described in detail with reference to Examples. Examples are provided only for the purpose of illustrating the present invention, and accordingly it is not intended that the present invention is limited thereto.

### EXAMPLE

### <Synthesis of Monomer>

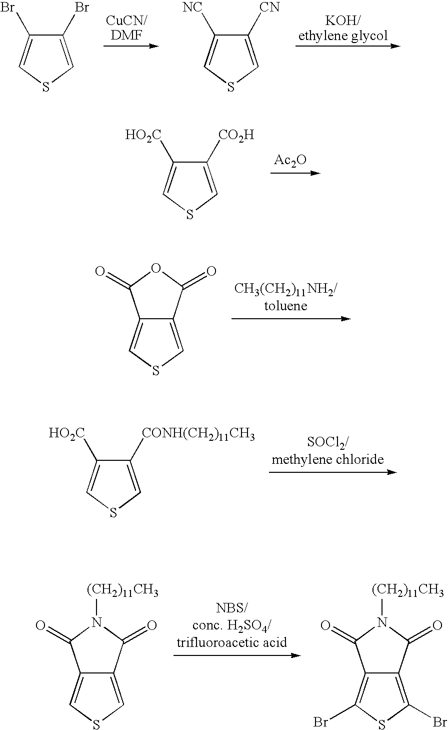

1) Synthesis of thiophene-3,4-dicarbonitrile

3,4-dibromothiophene (30 ml, 274 mmol) and copper cyanide (CuCN, 110 g, 1233 mmol) were aided to DMF (Dimethylformamide), and heated overnight under stirring. Then, the solution was cooled, and put into a FeCl₃ 6H₂O (432.5 g) solution dissolved in 2 M HCl (700 ml), followed by strong stirring for 1 hr at about 60° C. Then, impurities were filtered, and the mixture was extracted with methylene chloride three times. The obtained organic layer was washed with 6 M HCl, distilled water, a saturated NaHCO₃ aqueous solution, and distilled water in this order twice, and dried over MgSO₄. The dried mixture was column separated (hexane/THF=3/1) to obtain thiophene-3,4-dicarbonitrile (15.5 g, 42%).

2) Synthesis of thiophene-3,4-dicarboxylic acid

Thiophene-3,4-dicarbonitrile (13.4 g, 100 mmol) and KOH (56.1 g, 1 mol) were dissolved in ethylene glycol (167 ml), and heated under stirring overnight. The solution was cooled and put into distilled water, and washed with diethyl ether. The aqueous layer was oxidized with saturated hydrochloric acid, and organic materials were extracted with ethyl acetate. The organic layer was dried over MgSO₄ and the solvent was evaporated Then, recrystallization was performed in distilled water to obtain thiophene-3,4-dicarboxylic acid (15.2 g, 88%).

3) Synthesis of thiophene-3,4-dicarboxylic acid anhydride

Thiophene-3,4dicarboxylic acid (15.0 g, 87 mmol) was dissolved in acetic acid anhydride (218 ml), and heated under stirring overnight. After stirring, the solvent was evaporated, and recrystallization was performed in toluene to obtain thiophene-3,4-dicarboxylic acid anhydride (12.5 g, 93%).

GC/MS: [M+H]⁺=155

4) Synthesis of 4-dodecylcarbamoylthiophene-3-carboxylic acid

Thiophene-3,4-dicarboxylic acid anhydride (4.6 g, 30 mmol) and n-dodecylamine (6.0 g, 32 mmol) were dissolved in toluene, and heated under stirring overnight. The solution was cooled in a refrigerator, and then the formed solid was filtered The obtained solid compound was subjected to recrystallization in toluene to obtain 4dodecylcarbamoylthiophene-3-carboxylic acid (9.9 g, 97%).

GC/MS: [M+H]⁺=340

5) Synthesis of 5-dodecylthieno[3,4-c]pyrrole-4,6-dione

4-Dodecylcarbamoylthiophene-3-carboxylic acid (9.5 g, 28 mmol) was dispersed in methylene chloride (93 ml), and thionyl chloride (3.1 ml) was added dropwise for 10 min. The solution was heated under stirring overnight, cooled, and put into distilled water. The organic layer was separated, and washed with a 5% NaHCO₃ solution and distilled water. The resultant was dried over MgSO₄ and the solvent was evaporated, followed by recrystallization in hexane to obtain 5-dodecylthieno[3,4-c] pyrrole-4,6-dione (8.1 g, 90%).

GC/MS: [M+H]⁺=322

6) Synthesis of 1,3-dibromo-5-dodecylthieno[3,4-c]pyrrole-4,6-dione

5-Dodecylthieno[3,4-c]pyrrole-4,6dione (4.8 g, 15 mmol) were dissolved in sulfuric acid (24 ml) and trifluoroacetic acid (80 ml). Then, N-bromosuccinimide (10.7 g, 60 mmol) was divided into two portions, and addled The solution was stirred at about 50° C. overnight. After the solution was cooled, and put into iced water, and the organic layer was extracted with methylene chloride. The organic layer was washed with distilled water and 5% NaHCO₃, and dried over MgSO₄. The solvent was evaporated, and then column separated (n-hexane/THF=10/1), followed by recrystallization in ethanol three times to obtain 1,3-dibromo-5-dodecylthieno[3,4-c]pyrrole-4,6-dione (2.9 g, 40%).

GC/MS: [M]⁺=477

7) Synthesis of 2,2′-bis(trimethyltin)-5,5′-bithiophene

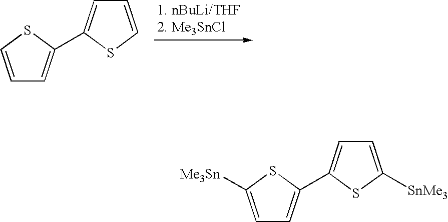

2,2′-Bithiophene (1.0 g, 6 mmol) was dissolved in THF (60 ml), and cooled to −78° C. Then, nBuLi (2.5 M in hexane, 5.3 ml) was added dropwise for 20 min. After stirring for a further 30 min, the solution was further stirred at room temperature for 1 hr. Trimethyltin chloride (2.6 g) dissolved in THF (10 ml) was added to the solution, and heated under stirring for 1 hr. The solution was cooled, and distilled water was put into the solution, followed by stirring for 10 min. Then, the organic layer was washed with distilled water and a 5% NaHCO₃ aqueous solution, and dried over MgSO₄. Re-crystallization was performed in acetonitrile three times to obtain 2,2′-bis(trimethyltin)-5,5′-bithiophene (1.3 g, 44%).

GC/MS: [M]⁺=492

### Example 1

### Synthesis of Compound Represented by Following Structural Formula

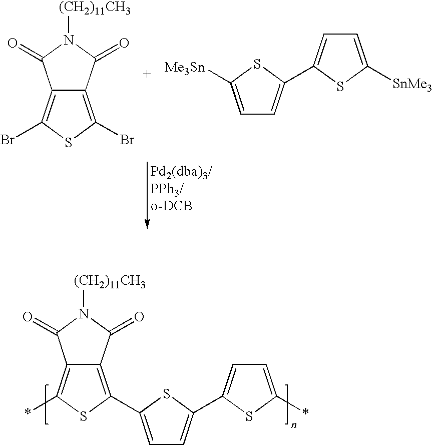

1,3-dibromo-5dodecylthieno[3,4-c]pyrrole-4,6-dione (240 mg, 0.5 mmol), 2,2′-bis(trimethyltin)-5,5′-bithiophene (246 mg, 0.5 mmol), Pd₂ (dba)₃ (9 mg, 2 mol %), PPh₃ ( 24 mg, 18 mol %), and 1,2-dichlorobenzene were put into a microwave vial, and the vial was placed into a microwave reactor (Biotage, Initiator™ 2.0 model). The conditions initial stirring (30 sec), temperature (220° C.), reaction time (10 min), power (normal)) were set, and the reaction was subjected Then, the reactant was diluted with chloroform (20 ml), and was aided dropwise to a solution of methanol/hyrochloric acid=10/1 (550 ml), followed by precipitation. After stirring for additional 1 hr, the precipitate was filtered, the resultant was washed with distilled water and methanol, and dried under vacuum. The obtained compound was extracted using a Soxhlet extractor to sequentially remove impurities of methanol (24 hr) and hexane (24 hr) to obtain a desired compound

### <Experimental Example>Production of Organic Thin Film Transistor

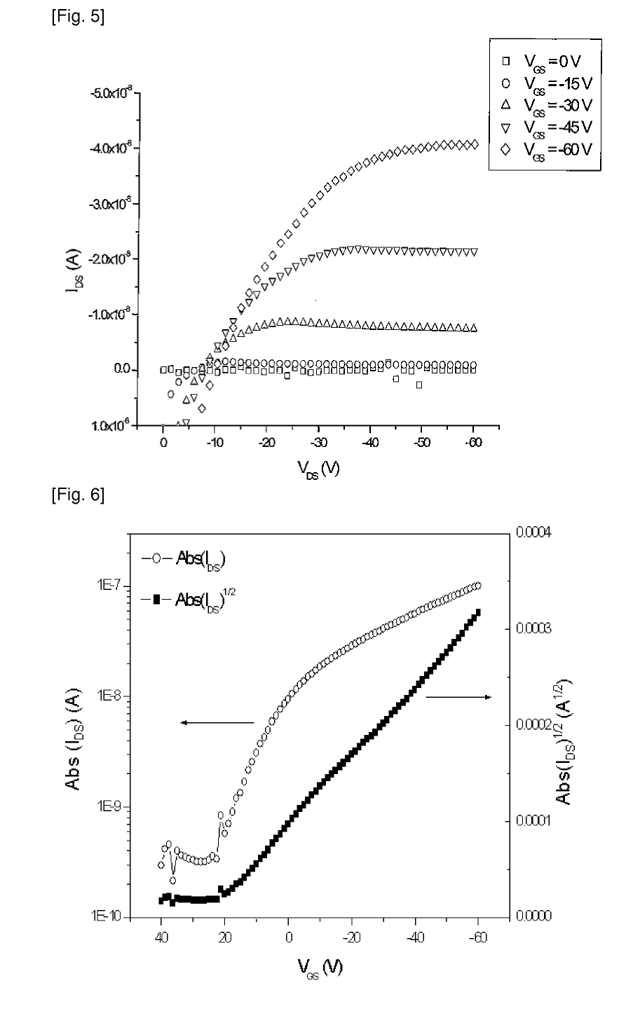

An n-type silicon wafer was used as a substrate and a gate electrode, and silicon oxide (300 nm) which was prepared by being grown with heat treatment thereon was used as a gate insulating film. Over the gate insulating film, an e-beam was used to form a source electrode and a drain electrode, made of gold The substrate prepared above was treated with HMDS (hexamethyldisilazane). Over the substrate, on which the source electrode and the drain electrode had been formed, the compound prepared in Example 1 which was dissolved in chloroform (0.5 w %) was spin-coated and heat-treated at 100° C. for 10 minutes to form an organic semiconductor layer. At this time, the width and the length of the channel of the organic thin film transistor were 1 mm and 100 μm, respectively. The charge mobility in the saturated region of the transistor prepared above was calculated to be 1.0×10⁻⁴ cm²/V.s. The results are shown in FIGS. 5 and 6.

FIG. 5 is a graph showing the change in drain-source current (I_{DS}) with respect to drain-source voltage (V_{DS}) in gate voltages, in which I_{DS} keeps in the saturated state above a specific value of V_{DS}, regardless of V_{DS}.

FIG. 6 is a graph showing the change in drain-source current (I_{DS}) with respect to gate voltage (V_{G}), in which the slope is sharply changed around V_{G}=20, indicating that switching performance is excellent.

1. A compound represented by the following Formula 1: 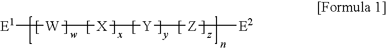
wherein W and Y are the same as or different from each other, and are each independently represented by the following Formula 2, 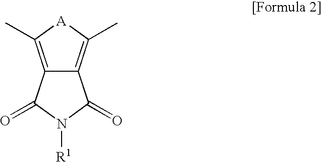
wherein R is the same as or different from each other, and is each independently a hydrogen atom; a halogen atom; an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA^{1═}CA²—, or —C≡C—, R′ and R″ are the same as or different from each other, and are each independently H, F, Cl, or CN, A¹ and A² are the same as or different from each other. and are each independently an alkyl group having 1 to 20 carbon atoms or aryl group,
A is O, S, Se, NR³, SiR³R⁴ ,or CR³R⁴, wherein R³ and R⁴ are the same as or different from each other, and are each independently a hydrogen atom; an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹CA²—, or —C≡C—, and R³ and R⁴ may be bonded to each other to form a ring, R′ and R″ are the same as or different from each other, and are each independently H, F, Cl, or CN, A¹ and A² are the same as or different from each other, and are each independently an alkyl group having 1 to 12 carbon atoms or aryl group having 1 to 12 carbon atoms,
In the Formula 1,
X and Z are the same as or different from each other, and are each independently —CA¹═CA²—; —C≡C—; an arylene group substituted with at least one R; or a heteroarylene group substituted with at least one R², R² is the same as or different from each other, and is each independently a hydrogen atom; a halogen atone an aryl group; a heteroaryl group; or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA¹CA²—, or —C≡C—, R′ and R″ are the same as or different from each other, and are each independently H, F, Cl, or CN, A¹ and A² are the same as or different from each other, and are each independently an alkyl group having 1 to 12 carbon atoms or aryl group, E¹ and E² are the same as or different from each other, and are each independently, a hydrogen atom; a halogen atom; an aryl group; a heteroaryl group; —Sn(R′R″R′″); —B(OR′)(OR″)₃; —CH₂Cl; —CHO; —CH═CH₂; —SiR′R″R′″; 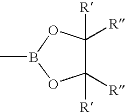
or a straight, branched, or cyclic alkyl group having 1 to 20 carbon atoms which is unsubstituted or substituted with F, Cl, Br, I, or CN, CH₂ groups in the alkyl group which are not adjacent to each other may be each independently substituted with —O—, —S—, —NH—, —NR′—, —SiR′R″—, —CO—, —COO—, —OCO—, —O—CO—O—, —S—CO—, —CO—S—, —CA^{1═}CA²—, or —C≡C—, R′, R″ and R′″ are the same as or different from each other, and are each independently H, F, Cl, or CN, A¹ and A² are the same as or different from each other, and are each independently an alkyl group having 1 to 12 carbon atoms or aryl group,
w, x, y, and z are mole fractions of W, X, Y, and Z, respectively,
w is a real number satisfying 0<w<1,
x is a real number satisfying 0<x<1,
y is a real number satisfying 0<y<1,
z is a real number satisfying 0<z<1, w+x+y+z=1, and
n is an integer of 1 to 10,000,
with the proviso that w=1, x=y=z=0, and when R¹ is an alkyl group, E¹ and E² are not hydrogen atom or halogen atom.

2. The compound according to claim 1, wherein A of Formula 1 is S.

3. The compound according to claim 1, wherein E¹ and E² of Formula 1 are the same functional groups.

4. The compound according to claim 1, wherein n of Formula 1 is 2 to 5,000.

5. The compound according to claim 1, wherein the compound represented by Formula 1 has a molecular weight of 1,000 to 500.000.

6. The compound according to claim 1, wherein the compound represented by Formula 1 is an alternating copolymer.

7. The compound according to claim 1, wherein X and Z of Formula 1 are each independently arylene groups or heteroarylene groups.

8. The compound according to claim 7, wherein the heteroarylene group contains one or more heteroatoms selected from the group consisting of a nitrogen atom, oxygen atom, sulfur atom and selenium atom.

9. The compound according to claim 7, wherein the arylene group or heteroarylene group is unsubstituted or substituted with one or more selected from the group consisting of F, Cl, Br, I, and CN, or a straight, branched, or cyclic alkyl group having 1 to 20 carbon atoms which is unsubstituted or substituted with F, Cl, Br, I, or CN.

10. The compound according to claim 9, wherein the CH₂ groups in the alkyl group which are not adjacent to each other are each independently unsubstituted or substituted with a substituent selected from the group consisting of —O—, —S—, —NH—, —NR′—, —SiR′R″—, —CO—, —COO—, —OCO—, —O CO—O—, —S CO—, —CO S—, —CA¹═CA²—, or —C≡C—: wherein R′ and R″ are the same as or different from each other, and are each independency H, F, Cl, or CN, A¹ and A² are the same as or different from each other, and are each independently an alkyl group having 1 to 20 carbon atoms or aryl group.

11. The compound according to claim 7, wherein the arylene group or heteroarylene group is a phenylene group, a phenylene group substituted with one or more nitrogen atoms, a naphthalene group, an alkyl fluorene group, an oxaaale group, a thiophene group, a selenophene group, or a dithienothiol)henie group.

12. The compound according to claim 1, wherein X and Z of Formula 1 are each independently selected from the group consisting of the following structural formulae: 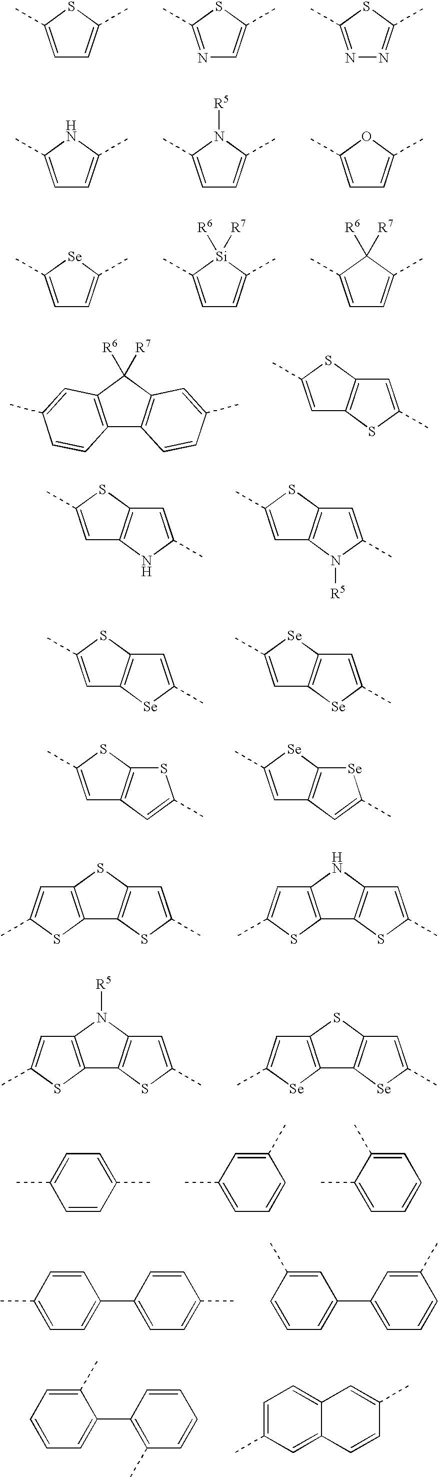 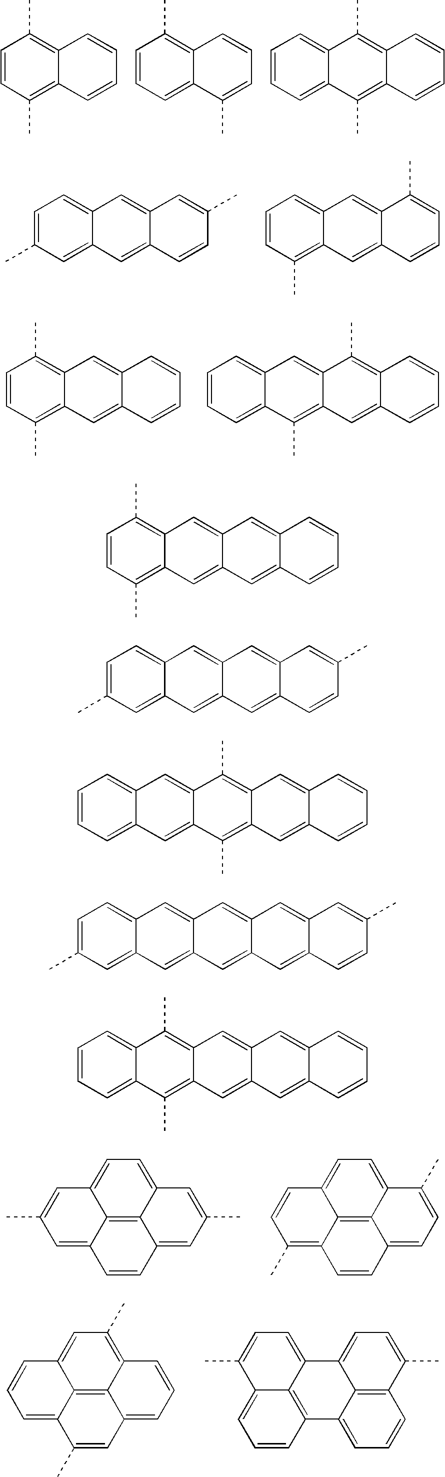 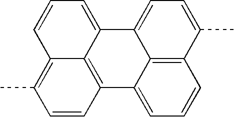
wherein R⁵, R⁶, and R⁷ are the same as or different from each other, and are each independently hydrogen atom, alkyl group, or aryl group.

13. The compound according to claim 1, wherein R¹, R², R³, R⁴, E¹, and E² of Formula 1 are each independently selected from the group consisting of phenyl group, naphthyl group, anthracenyl group, pyrenyl group, pherylenyl group, pyridyl group, bipyridyl group, carbaaale group, thiophenyl group, qiinolinyl group, isoqimnolinyl group, furyl group, pyridyl group, pyrrolyl group, and phenanthryl group.

14. The compound according to claim 1, wherein Formula 1 is represented by any one of the following Formulae 3 to 12: 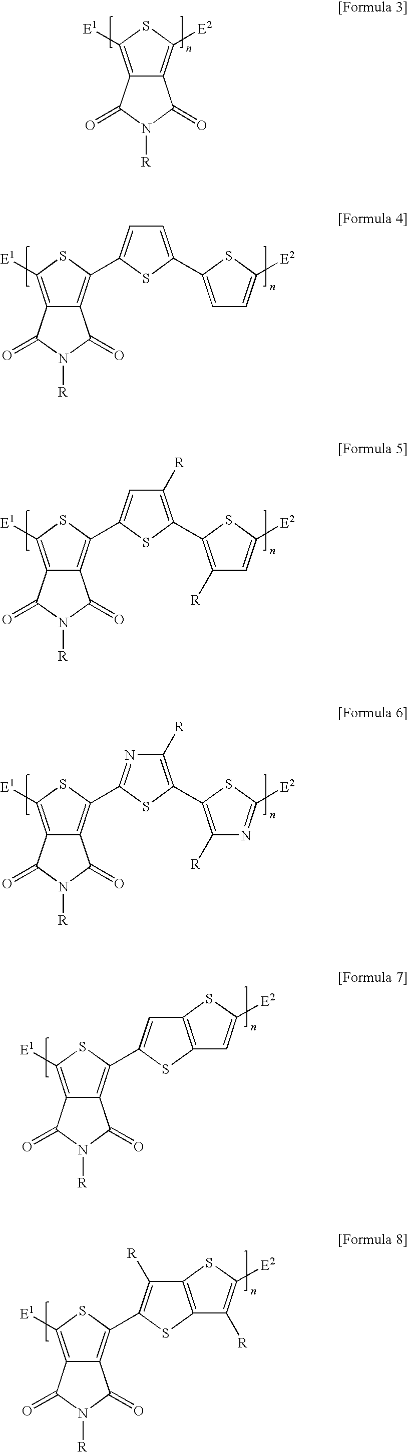 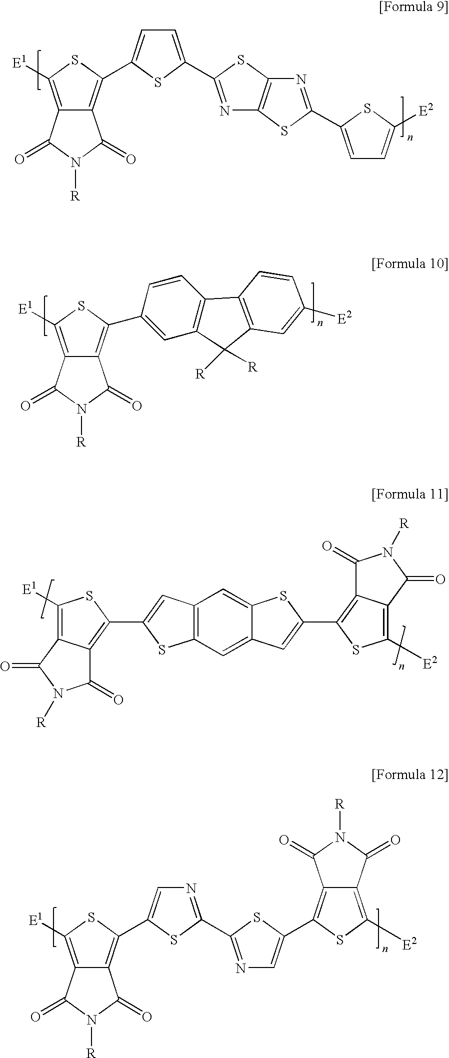
wherein Rs are the same as defined in R¹ of Formula 1, and are the same as or different from each other in the same molecule, and
n, E¹ , and E² are the same as defined in Formula 1.

15. A method for preparing the compound of claim 1, comprising the step of reacting a halogen substituent of W or Y and a material having a structure represented by X or Z by Stille coupling, Kumada coupling, or Suzuki coupling: wherein W, Y, X, and Y are the same as defined in Formula 1.

16. An organic electronic device using the compound according to claim 1 as an organic semiconductor material.

17. The organic electronic device according to claim 16, wherein the organic electronic device is an organic light emitting device.

18. The organic electronic device according to claim 16, wherein the organic electronic device is an organic thin film transistor.

19. The organic electronic device according to claim 16, wherein the organic electronic device is an organic solar cell.
